(19) United States
(12) Patent Application Publication
(10) Pub. No.: US 2024/0325708 A1
(43) Pub. Date: Oct. 3, 2024

(54) CONNECTOR FOR CATHETER AND LABEL PLATE THEREFOR

(71) Applicant: BIOTRONIK AG, Bülach (CH)

(72) Inventors: Luc von Orelli, Zuerich (CH); Matteo Taormina, Eglisau (CH)

(21) Appl. No.: 18/439,094

(22) Filed: Feb. 12, 2024

(30) Foreign Application Priority Data
Mar. 31, 2023 (EP) 23165785.9

(51) Int. Cl. A61M 39/10; G09F 3/00

(52) U.S. Cl. CPC A61M 39/10; G09F 3/0295

(57) ABSTRACT

A label plate for a catheter connector includes a label plate body configured to be mounted around (and can be fixed onto) an outer shell surface of the catheter connector. A shape of an inner surface of at least a segment of the label plate body corresponds to the shape of an outer surface of at least a section of the catheter connector.

### PRIORITY CLAIM

This application claims priority under 35 U.S.C. § 119 and all applicable statutes and treaties from prior European Application No. EP23165785, which was filed Mar. 31, 2023.

### FIELD OF THE INVENTION

A filed of the invention concerns catheters and its connectors.

### BACKGROUND

Catheters are medical devices that include an elongated, usually flexible shaft made of medical grade materials and serve a broad range of functions. Normally, they are inserted into a patient's body for diagnosis and/or treatment. For example, the catheter can be inserted into a body cavity, duct, or vessel, brain, skin or adipose tissue. Functionally, a catheter can allow drainage, administration of fluids or gases, access by surgical instruments, or sampling of compounds from the patient's body. One important catheter type is a balloon catheter that has an inflatable balloon at its distal end and is used to expand/deploy a stent or similar implant or to enlarge a narrow opening or passage within the patient's body. Balloon catheters are used, for example, in angioplasty or balloon septostomy, e.g. via cardiac catheterization, tuboplasty via uterine catheterization or pyeloplasty.

In order to provide its specific function, in many cases the catheter needs to be connected to a specific device (e.g. inflation device) or reservoir by at least one male or female connector (fitting) which is typically provided at or near a proximal end of the elongated shaft. For correct usage, the catheter's connector is connected to the specific device or reservoir that fits to the specific function and size of the catheter and/or connector. To assist the health care professional (HCP) in choosing the correct catheter (e.g. among different catheters in use), the connector can include a specific marking that is provided, for example, at a handle-like section or on a surface of the connector. This marking can be difficult to read because a typical catheter connector is transparent and small. Colored materials can make proper connector and catheter matching easier, but colorizing additive can change properties of the plastic material often used for the connector. Use of different colored connectors also increases costs due to extensive warehousing for catheters having different colors.

### SUMMARY OF THE INVENTION

A preferred label plate for a catheter connector includes a label plate body configured to be mounted around (and can be fixed onto) the outer shell surface of the catheter connector. A shape of a bottom side or an inner surface of at least a segment of the label plate body corresponds to the shape of an outer surface of at least a section of the catheter connector.

### BRIEF DESCRIPTION OF THE DRAWINGS

The present invention will now be described in further detail with reference to the accompanying schematic drawings, wherein

FIG. 1 shows a first embodiment of a label plate and a first embodiment of a catheter in a perspective side view during manufacturing,

FIG. 2 depicts the first embodiment of a label plate fixed to a second embodiment of a catheter in a perspective side view in a fully mounted state,

FIG. 3 shows the system of FIG. 2 in a different perspective side view in a fully mounted state,

FIG. 4 shows a second embodiment of a label plate and the catheter of FIG. 1 in a perspective side view during manufacturing,

FIG. 5 depicts a third embodiment of a label plate and the catheter of FIG. 1 in a perspective side view during manufacturing,

FIG. 6. depicts a fourth embodiment of a label plate and the catheter of FIG. 1 in a perspective side view during manufacturing,

FIG. 7 depicts a fifth embodiment of a label plate and the catheter of FIG. 1 in a perspective side view during manufacturing,

FIG. 8 shows a sixth embodiment of a label plate and the catheter of FIG. 2 in a perspective side view in a fully mounted state,

FIG. 9 depicts a seventh embodiment of a label plate and the catheter of FIG. 2 in a side view in a fully mounted state, and

FIGS. 10-12 depict the usage of a guide wire insertion tool for guide wire introduction in side views.

### DETAILED DESCRIPTION OF THE PREFERRED EMBODIMENTS

A label plate according to the invention includes configured to firmly or releasably attach around a catheter connector. A firm attachment of the label plate to the catheter connector can be a firm-bond in the sense of a material bond or a firm form or force bond which is only releasable by destruction of the label plate body or the catheter connector. This is important, because the label plate is used to mark the connector and/or the catheter without changing the composition of the connector, for example, to show the type and size of the connector and/or catheter. For that, the marking can be provided and represented by the properties of the label plate body itself (its color, form, size, surface structure or similar) and/or can be printed or written to the surface of the label plate body. The label plate body can include or consist of a material that is different from the material of the connector. Unlike the material of the connector, which is adjusted and chosen to its specific medical purpose, the material of the label plate body can be chosen and adjusted to realize a better readable marking (e.g. by the color of the material or by being better markable by a marking, i.e. writing, method). The label plate can further be stored separately from the catheter and its connector and can be assigned and fixed to the outer shell surface of the respective connector shortly prior to sterilization (during manufacturing), only. For a firm-bond in the sense of a material bond, the label plate body can, for example, be glued or laser welded to the catheter connector.

That the shape of a bottom side or inner surface of at least a segment of the label plate body corresponds to the shape of an outer surface of at least a section of the catheter connector (e.g. of the handle-like section) means that the side or surface of the label plate body that is configured to abut the outer surface of the connector corresponds to the shape of this surface. Accordingly, the correct position of the label plate at the connector is easily and quickly adopted. Further, thereby the volume occupied by the label plate at the connector is small. Additionally, the label plate body can correspond to the shape of the outer surface of the catheter connector, as well.

The term “connector” as it is used in the present description refers to a component provided at the proximal end of the catheter. The connector can include a plurality of sections, for example the actual connector section (i.e. in the literal sense) where the catheter is connected with the connector's counterpart provided by a device or a reservoir to be connected with the catheter, a handle-like section where the user (HCP) grips the connector to attach the connector to its counterpart and a transition section where the connector is attached to the elongated and flexible catheter shaft. The connector section in the narrower sense and the handle-like section can be rigid, while the transition section can have an intermediate flexibility between the rigidity of the rigid sections and the flexibility of the adjacent catheter shaft. The label plate can be fixed to at least one of the above sections of the connector. In one embodiment, the label plate can be fixed to the handle-like section. In one embodiment, the connector section in the narrower sense is the most proximal section and adjacent to the handle-like section and the transition section is the most distal section of the connector (in the broad sense). The connector includes at least one opening extending from the proximal end to the distal end that goes through.

The label plate provides a marking to the catheter and/or its connector in an easy and cost-effective way. A further advantage is that the label plate can be configured such that the area where a making is provided can be designed much greater than the underlying area of the connector. Accordingly, the label plate can increase the surface available for marking so that the marking can contain more information for the HCP. The marking can include numbers, letters and picture-like marking such as pictograms or barcodes, RFID tags as well as trademark signs and similar marking. For example, the marking can contain (e.g. in case the catheter is a balloon catheter) the nominal pressure (NB), the rated burst pressure (RBP), and/or the maximum allowed diameter (MAD). The label plate (in manufacturing) can be stored separately from the catheter or the connector and can consist of a nontransparent material that allows marking with a greater contrast that is easier to read. In particular, a coloration or specific form of the label plate allows fast identification of the correct catheter and/or connector in a stressful clinic environment. A further advantage of such label plate is that its look, and feel can be adapted to the user's, authorities' and market's requirements quickly and without any need (or reduced requirements for passing regulatory admission process) as the label plate does not change the interior construction or material of the connector. It is just fixed to the outer surface of the connector.

In one embodiment, the label plate or the shape of the label plate is configured such that it allows exposure of a central portion of the catheter connector that extends along a longitudinal axis section of the catheter connector and/or a lateral axis section of the catheter connector. In some embodiments, the label plate body can include a through-hole and/or a recess, wherein the through-hole and/or the recess are configured to be arranged above the central portion of the catheter connector and to extend along the longitudinal axis section of the catheter connector and/or the lateral axis section of the catheter connector in a state fully mounted to the catheter connector. In addition or alternatively, the shape of the label plate can be of a dimension that at least part of the central portion of the catheter connector that extends along a longitudinal axis section of the catheter connector and/or the lateral axis section of the catheter connector is not covered by the label plate when fixed to the outer shell surface of the catheter connector. In addition or alternatively, the label plate can include a window section including and/or being made of a transparent material and being arranged at the label plate so that it reveals the central portion of the catheter connector that extends along a longitudinal axis section of the catheter connector and/or a lateral axis section of the catheter connector when the label plate is fixed to the catheter connector.

The label plate or the shape of the label plate is preferably configured to allow exposure of a central portion of the catheter connector that extends a longitudinal axis section of the catheter connector to allow the HCP to observe the interior of the connector in the area of the connector's longitudinal axis. Thereby, the HCP can control whether the connector/catheter operates correctly during the patient's treatment or diagnosis. Such shape can provide a window-like or slit-like opening through which the HCP can observe the central portion of the catheter. The longitudinal axis/direction of the connector is the extension of the longitudinal axis/direction of the catheter shaft. A lateral axis of the connector is any axis which forms an angle unequal to 0° with the longitudinal axis of the catheter connector. The central portion is a portion of the connector that extends along and parallel the longitudinal axis/direction, respectively, the lateral axis and the through opening. This portion can include only part of the width (greater extension perpendicular to the longitudinal axis) of the connector but the central part of the width so that the HCP can observe the fluid or other material running along the through opening of the connector. The central portion can include only a part of the full length of the connector (extension along the longitudinal axis).

In one embodiment, the at least one segment has a U-shape in a state fully mounted to the catheter connector, wherein the U-shape provides an intermediate space (slit-like opening) to allow exposure of a central portion of the catheter connector that extends along the longitudinal axis section of the catheter connector. In another embodiment, the at least one segment has a frame-like shape in a state fully mounted to the catheter connector, wherein the frame-like shape provides window-like opening to allow exposure of a central portion of the catheter connector that extends along a longitudinal axis section of the catheter connector. The intermediate space or window-like opening is an open space located between the two legs of the U-shape. In one embodiment, the connector's central area along its longitudinal axis is covered by the label plate body only in or near the base section of the “U” or “frame”—where the two legs or sides of the frame are connected.

In one embodiment, the U-shaped segment is formed by at least one first plate-like leg, at least one second plate-like leg and at least one plate-like web connecting one of the at least one first leg and one of the at least one second leg. In this embodiment the open intermediate space is formed between the first and the second leg, wherein the web can cover a section of the longitudinal axis of the connector when it is fully mounted to the connector. In one embodiment, the web is located on the same side of the connector as the first and second plate-like legs. In an alternative embodiment, the web is located on the opposite side of the connector as the first and second legs.

The fixing of the label plate to the connector is provided by features of the label plate body or can include other types of fixation. Fixation features can be separate from and/or adjacent to the label plate body and/or can be integrated within the label plate body. In one embodiment, the label plate body is configured such that it provides a form-fitting connection, a force-fitting connection and/or a firmly bonded connection with the catheter connector. For example, the label plate body can include at least two protrusions that clamp onto the outer rim of the connector forming a snap-fit connection. Alternatively, such protrusions can interact with at least two protrusions at the opposite side of the label plate body forming a snap-fit connection so that the connector is arranged between an upper segment of the label plate body and a lower segment of the label plate body. Additionally, or alternatively, the label plate body can include an adhesive at one side to attach the label plate to the surface of the connector. Preferred label body fixation features can include protrusions that clamp around the connector, a bent transition section that clamps onto the connector, and projections that form a snap-fit connection with each other around the connector.

In one embodiment, a material located at the upper side or outer surface of the label plate body (i.e. a surface that points away from the connector) is writable or printable. Accordingly, the desired marking can be provided in an easy and cost-effective way. For example, the material located at the upper side or outer surface of the label plate body can include laser sensitive particles or pigments for laser marking. Alternatively, inkjet printing can be used for printing the desired information to the label plate body. In this case, the material at the outer surface or outer side of the label plate body is configured such that ink or toner used for inkjet printing can adhere to the surface/side. In one embodiment, the printing ink or toner can be additionally adhered to the surface of the label plate by a cover layer covering the printed marking with a thermal or curable material that can be toughened or hardened after printing by a thermal or UV treatment. Alternatively, the printing ink or toner itself can be cured by thermal or UV treatment.

In one embodiment, the label plate body includes an appendage or similar projection providing a secondary function, wherein the appendage (e.g. a projection) is, for example, formed as a guide wire insertion tool. The guide wire insertion tool can ease the introduction of the guide wire into a catheter tube as explained in more detail below. The appendage can be removed from the label plate prior the secondary function is started or executed. For that, a predetermined breaking point can be provided between the label plate and the appendage for easy removal.

In one embodiment, the label plate body includes two opposite segments, wherein at least one segment of the two segments includes the bottom side or inner surface with the shape which corresponds to the shape of the outer surface of at least a section of the catheter connector, e.g. a U-shape, wherein both segments are connected by a hinge. Such hinge allows correct relative movement (folding) of two opposite segments prior the label plate adopts its fully mounted state, respectively, is connected to the catheter connector. In one embodiment, the catheter connector is located between the first segment and the opposite second segment in the fully mounted state. In one embodiment, both, the first and the second segment have a U-shape.

In one embodiment, the label plate body includes a predetermined breaking point. As indicated above, such predetermined breaking point can allow removal of an appendage with a secondary function from the label plate. Alternatively or additionally, such breaking point can indicate to the user if the label plate has been removed from the connector without authorization.

In one embodiment, an RFID tag is embedded within the label plate body material. The RFID tag can include a microchip for storing and processing information and production of the RF signal, an antenna for receiving and transmitting the RF signal and a substrate.

The above object is further solved by a system including a catheter connector and a label plate as described above, wherein the shape of a bottom side or inner surface of the label plate body corresponds to the shape of the outer surface of a section of the catheter connector. The connector is a connector in a broader sense as explained above. The system has the advantages that are described above with regard to the label plate. It is therefore referred to the above discussion.

The above object is further solved by a system including a catheter having an elongated, flexible shaft and a connector at the proximal end of the shaft, wherein the system further includes a label plate as described above, wherein the shape of a bottom side or inner surface of the label plate body corresponds to the shape of the outer surface of a section of the catheter connector. The connector is a connector in a broader sense as explained above. The system has the advantages that are described above with regard to the label plate. It is therefore referred to the above discussion.

In one embodiment of both above systems, the catheter connector section (in the narrower sense) of the connector is a male or female Luer connector. Such connector is often used for attachment in the medical field for providing leak-free connections. The Luer connector is used to form a Luer taper together with its respective mating connector.

In one embodiment of the system including the catheter, the catheter is a balloon catheter and includes an inflatable balloon at the distal end of the catheter shaft.

With respect to all above embodiments, the connector (in the broader sense) can include or consist of a material of the group of thermoplastics, the label plate can also include or consist of a material of the group of thermoplastics. Further the label plate or each segment of the label plate can have a thickness of about 0.2 to 5 mm, preferably of about 0.2 to 2 mm. The surface area for writing/printing at the label plate can be greater than 5 mm×15 mm=75 mm². The label plate can be produced by injection moulding or 3D printing.

The catheter can include, for example a single or double connector at its proximal end, an inflatable/deflatable balloon at its distal end and a flexible, elongated shaft in between having a longitudinal axis. Within a distal section of the shaft, the tube can include two lumen wherein a first lumen is the inflation lumen that is in fluid communication with the balloon and a guide wire lumen. The guide wire lumen can be provided by an inner tubular member whereas the inflation lumen can be provided by an outer tubular member. The guide wire can run along an outer surface of the catheter shaft within a proximal section of the catheter shaft.

In the following, the invention is described with regard to balloon catheters. Generally, the invention can also be applied to other catheter types so that the following examples are not intended to limit the scope of the disclosed subject matter in any manner, in particular not to balloon catheters.

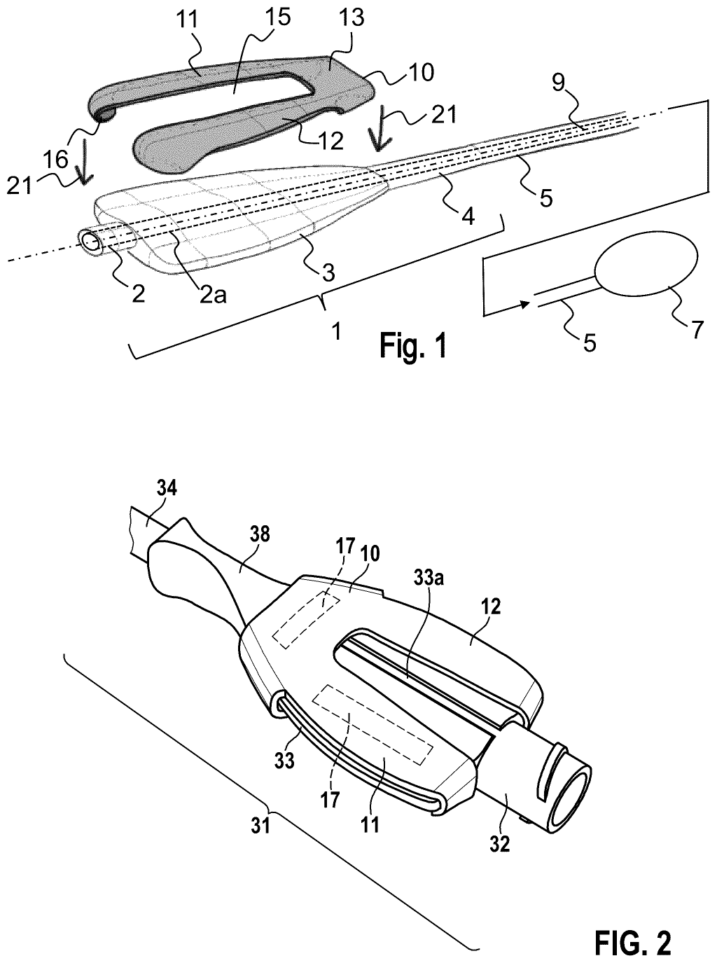

FIG. 1 shows a balloon catheter including a tubular shaft 5 that is connected at its proximal end to a connector 1 and at its distal end to a balloon 7. The tubular shaft 5 and the connector 1 have a longitudinal axis 9. The shaft 5 includes an inflation lumen, wherein the balloon 7 is in fluid communication with the inflation lumen. The inflation lumen extends from the connector 1 to the balloon 7. At the distal end the shaft 5 includes a guide wire lumen that runs parallel to the inflation lumen and through the balloon 7. A guide wire extends through this guide wire lumen during operation of the catheter for guiding the catheter to the treatment/diagnosis location.

The connector 1 includes a connector section 2 at its distal end and a transition section 4 at its proximal end. A flat handle-like section 3 is provided in longitudinal direction between the connector section 2 with, e.g. a male Luer connector and the transition section 4. The transition section 4 forms the transition to the flexible shaft 5 and a fluid tight connection to the shaft 5 and is more flexible than the rigid connector section 2 and handle-like section 3. The handle-like section 3 can be gripped by the HCP to connect the catheter to a pre-determined device or reservoir having a connector counterpart (e.g. a female Luer connector). The connector 1 is formed of transparent plastic material so that the HCP can observe the fluid running a long an opening 2a going through the connector 1.

The system further includes a label plate 10 with a U-shaped body having a first plate-like leg 11, a second plate-like leg 12 and a plate-like web 13 connecting the first leg 11 and the second leg 12 at their distal ends. Between the first leg 11 and the second leg 12 there is an intermediate space 15 that allows exposure of a central portion of the connector 1 located at and around the longitudinal axis 9 of the connector 1. Each of the first leg 11 and the second leg 12 includes a protrusion at its proximal end that is bent such that it provides a form-fitting connection around the handle-like section 3 of the connector 1. Additionally, similar protrusions are provided at the ends of the web 13 extending transverse to the longitudinal axis 9. The label plate 10 can be firmly attached to the section 3 of the connector 1 by clamping (see arrows 21) using the protrusions 16 that engage around the rim of section 3 of the connector 1 in the fully mounted state.

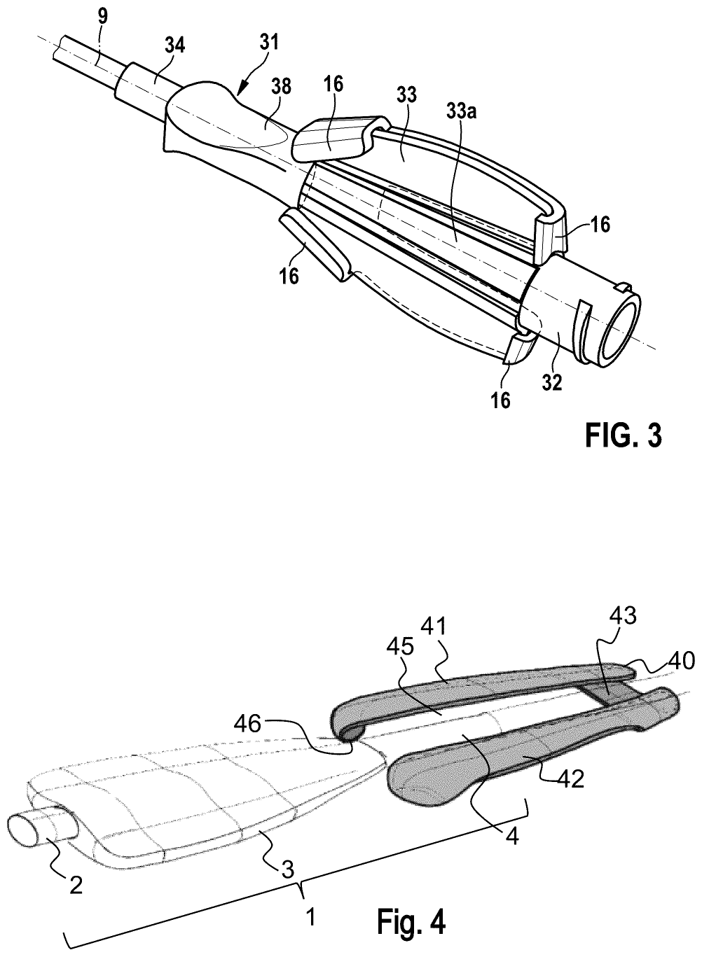

A similar attachment and label plate 10 is shown in FIGS. 2 and 3 in a fully mounted state. The connector 31 of the catheter is different from the one depicted in FIG. 1 in that the connector 31 includes a fourth section between the transition section 34 and the first handle-like section 33 in longitudinal direction-namely a second handle-like section 38. The second handle-like section 38 is smaller than the first handle-like section 33. The upper flat surfaces of the first and the second handle-like sections 33, 38 are twisted by approximately 90°. The connector 31 further includes a connector section 32 and a transition section 24 similar to the catheter connector 1 shown in FIG. 1. In FIGS. 2 and 3 it is clearly shown that the intermediate space 15 of the U-shaped label plate body exposes a central portion 33a of the first handle-like section 33 of the connector 31 for observation of the through opening (analogous to opening 2a of connector 1 shown in FIG. 1). Further, FIG. 3 depicts how the protrusions 16 encompass the rim of the first handle-like section 33. As one can derive from FIGS. 1 to 3, the lower side of the label plate 10 abutting to the upper surface of the handle-like section 3, 33 has a shape that corresponds to the upper surface of the handle-like section 3, 33. Thereby, the thickness of the respective handle-like section 3, 33 is only slightly increased compared with the thickness of the handle-like section 3, 33 without label plate 10.

FIG. 2 further shows that label plate 10 has a color different from the color of the connector 31. Further, as it is not transparent, the marking 17 has a clear contrast with regard to the label plate body and thereby easy readable.

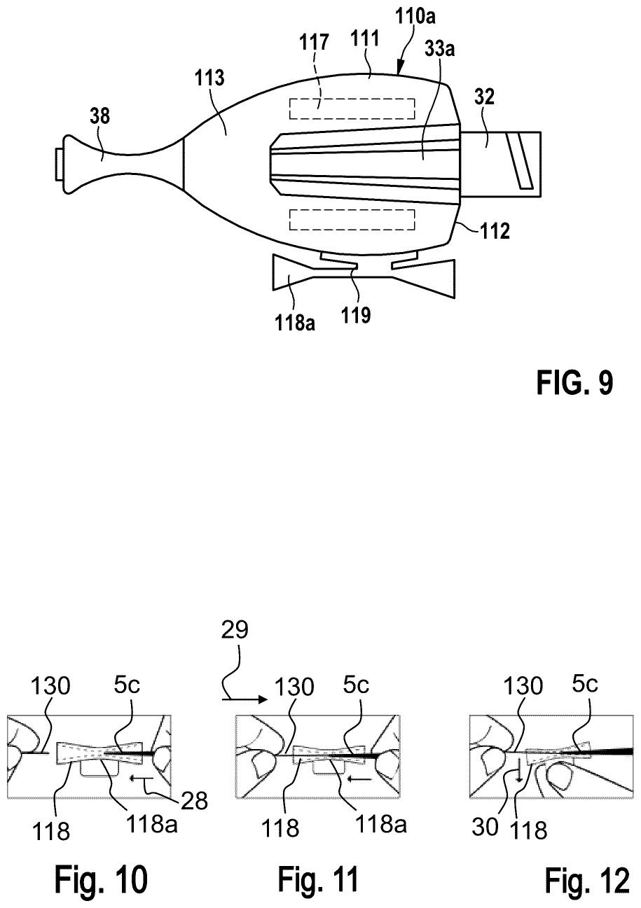

The embodiments of the system shown in FIGS. 4 to 7 refer to a catheter and connector 1 as shown in FIG. 1 with different embodiments of a label plate. The embodiments of FIGS. 8 and 9 refer to a catheter and connector 31 as shown in FIGS. 2 and 3 with further embodiments of a label plate.

The label plate 40 of FIG. 4 is quite similar to the label plate 10 depicted in FIGS. 1 to 3 as it has similar first leg 41 and second leg 42 as well as protrusions 46 at the proximal end of legs 41, 42. However, web 43 connecting the first leg 41 and the second leg 43 is located at the lower side so that the handle-like section 3 is located between the legs 41, 42 on one side and the web 43 at the other side. Thereby the label plate 40 is clamped to the handle-like section 3 when the label plate 40 is fully mounted to the connector 1.

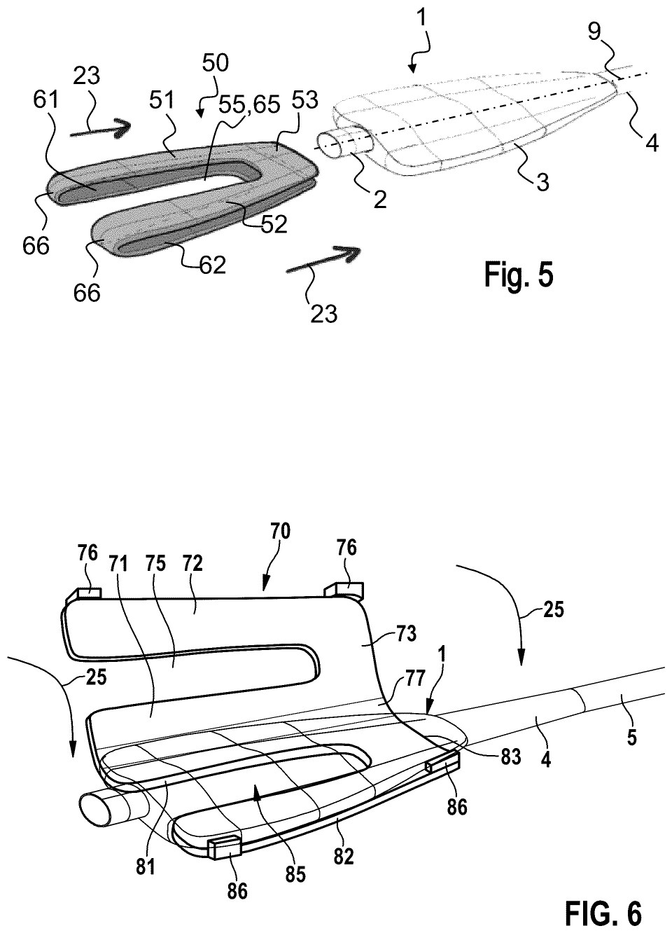

The label plate 50 shown in FIG. 5 includes a first U-shaped segment including a first leg 51, a second leg 52 and a web 53 similar to the embodiment of FIG. 1 and an opposite, second U-shaped segment including a first leg 61, a second leg 62 and a web 63. Both segments form an intermediate space 55, 65 between legs 51, 52 and 61, 62, respectively. Both segments are connected by a bent transition section 66 at the distal end of the legs 51, 52, 61, 62, wherein the first leg 51 of the first segment is connected to the first leg 61 of the second segment and the second leg 52 of the first segment is connected to the second leg 62 of the second segment. For attachment to the connector 1 the label plate 50 is moved along the longitudinal axis in distal direction (see arrows 23). The connector 1 is clamped between the first and the second segments and by the transition section 66 so that the label plate 50 is thereby firmly fixed to the connector 1.

The label plate 70 depicted in FIG. 6 includes two U-shaped segments which clamp the connector 1 therebetween, as well. Here, a first segment having a first leg 71, a second leg 72, an intermediate space 75 and a web 73 is folded along a hinge-like connection area 77 running parallel to the longitudinal axis 9 of the connector 1. The second segment has a first leg 81, a second leg 82, an intermediate space 85 and a web 83. The connection area 77 connects the first leg 71 along its full length (in longitudinal direction) to the first leg 81 of the second segment. During manufacturing, the label plate 70 is folded along the connection area 77 (see arrows 25) and firmly fixed to the connector 1 using two protrusions 76, 86 at each segment located at a rim of the segment opposite the connection area forming snap-fit connections.

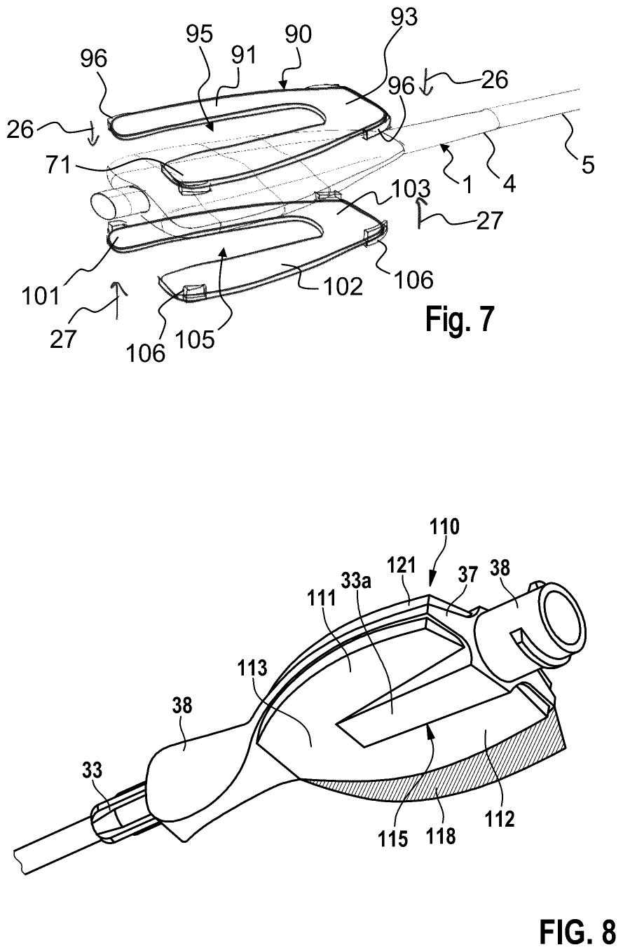

The label plate 90 shown in FIG. 7 includes initially two separate U-shaped segments each with a first leg 91, 101, a second leg 92, 102 and a web 93, 103 connecting the first leg 91, 101 to the second leg 92, 102. At each one of the four outer corners of the segments, the segments include a projection 96, 106 that is configured such that it forms a snap-fit connection with the opposing one 106, 96 when both segments are fully mounted to the connector 1. For that, each segment is moved towards the other one perpendicular to the longitudinal direction (see arrows 26, 27). In this embodiment, the connector 1 is located between the first segment and the second segment of the label plate 90, as well.

The label plate 110 (see FIG. 8) is similar to the embodiment shown in FIG. 7 since it has a first segment with a first leg 111, a second leg 112 and a web 113. The second segment has a first leg 121, a second leg and a web, as well. The label plate includes a section 118 that increases the size of the area formed by the second leg 112. The section 118 can also be integrated to label plates of the embodiments shown in FIGS. 1-6.

The similar embodiment of a label plate 110a depicted in FIG. 9 includes a guide wire insertion tool 118a (instead of a section 118) that is provided on the rim of the second leg of this embodiment. This guide wire insertion tool 118a can be removed from the label plate 110a by breaking the plastic material at a pre-determined breaking point 119 at a web that connects the tool 118a to the second leg 112. Then, the tool 118a can be used as shown in FIGS. 10 to 12 to insert a guide wire 130 into the distal end of the guide wire lumen. For that the distal tip 5c of the catheter is introduced into a through going passage of the tool 118 on one side (see FIG. 10) and the guide wire 130 on the other side (see FIG. 11). As the inner passage is reduced in its diameter at the central section of the tool 118a, the guide wire 130 is led to the opening of the distal tip 5c. This can be eased by slightly rotating the tool 118a (see arrow 30 in FIG. 12) so that the guide wire 130 is guided by the inner surface of the opening to the reduced diameter section.

The above description explains different embodiments of label plates which are cost-effective means for marking the connector/catheter, for example by laser printing or ink-jet printing.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

1. A label plate for a catheter connector comprising a label plate body configured to be mounted around outer shell surface of the catheter connector, wherein a shape of an inner surface of at least a segment of the label plate body corresponds to a shape of an outer surface of at least a section of the catheter connector.

2. The label plate of claim 1, wherein the label plate body comprises an intermediate space configured for exposure of a central portion of the connector.

3. The label plate of claim 2, wherein the label plate body comprises a U-shape and the intermediate space is defined in a middle of the U-shape.

4. The label plate according to claim 3, wherein the U-shape comprises at least one first plate-like leg, at least one second plate-like leg, and at least one plate-like connecting web the at least one first plate-like leg and the at least one second plate-like leg.

5. The label plate according to claim 1, wherein the label plate body is configured to define a form-fitting connection, a force-fitting connection and/or a firmly bonded connection with the catheter connector.

6. The label plate according to claim 1, comprising a writable or printable material located at the upper side or outer surface of the label plate body.

7. The label plate according to claim 1, wherein the label plate body comprises an appendage providing a secondary function.

8. The label plate according to claim 7, wherein the appendage comprises a guide wire insertion tool.

9. The label plate according to claim 1, wherein the label plate body comprises two opposing segments, wherein both segments are connected by a hinge.

10. The label plate according to claim 1, wherein the label plate body comprises a predetermined breaking point.

11. The label plate according to claim 1, comprising an RFID tag embedded within the label plate body.

12. A catheter system, comprising a catheter connector and a label plate (according to claim 1.

13. A catheter system comprising a catheter having an elongated, flexible shaft and a catheter connector at the proximal end of the shaft (5), wherein the system further comprises a label plate according to claim 1, the label plate being mounted around the catheter connector.

14. The system of claim 13, wherein the catheter connector is a Luer connector.

15. The system of claim 13, comprising an inflatable balloon at a distal end of the catheter shaft.
